(19) United States
(12) Patent Application Publication
(10) Pub. No.: US 2008/0153796 A1
(43) Pub. Date: Jun. 26, 2008

(54) Promotion of wound contraction

(75) Inventors: Nick Occleston, Manchester (GB); Kerry Nield, Manchester (GB); Sharon O'Kane, Manchester (GB); Mark Ferguson, Manchester (GB)

Correspondence Address: NIXON & VANDERHYE, PC, 901 NORTH GLEBE ROAD, 11TH FLOOR, ARLINGTON, VA, 22203, US

(73) Assignee: RENOVO LIMITED, Manchester (GB)

(21) Appl. No.: 11/723,194

(22) Filed: Mar. 16, 2007

(30) Foreign Application Priority Data
Dec. 23, 2006 (GB) 0625964.2

(51) Int. Cl. A61K 31/56; A61P 17/02

(52) U.S. Cl. 514/182

(57) ABSTRACT

The present invention relates to medicaments and methods for promoting wound contraction. In certain embodiments the invention provides medicaments and methods capable of promoting wound contraction without acceleration of re-epithelialisation.

The present invention relates to medicaments and methods for promoting wound contraction. In certain embodiments the invention provides medicaments and methods capable of promoting wound contraction without acceleration of re-epithelialisation.

The wound healing response proceeds through a number of over-lapping processes that bring about repair of the damaged area. Briefly, the processes involved include: the inflammatory response; the development of granulation tissue; wound contraction; re-epithelialisation; and scar remodelling.

Wounds are a source of discomfort to those afflicted, and may be associated with, or give rise to, a number of clinical difficulties or complications.

Wounds are painful, even aside from the events associated with their formation, and delays in the healing of wounds may be associated with extended incidences of pain to the sufferer. Wounds can also decrease the mechanical function of the injured area.

The continued presence of open wounds can also be associated with many clinical problems, including blood loss and the possible incidence of infection.

In the light of the above, it will be seen that the ability to promote healing of wounds is advantageous for many different reasons. The ability to promote wound contraction, and thereby reduce wound size and many of the disadvantages associated with wounding, would provide many advantages in clinical management of wounds.

Although the need for medicaments and methods capable of promoting contraction, and thus closure, of wounds is recognised by those skilled in the art, there remains a lack of widely applicable therapies that may be used to achieve this end. Accordingly, there is a well recognised requirement for new, alternative, and more effective, medicaments and methods by which such acceleration may be attained.

Currently the control of the wound closure is reliant upon the initial dressing and wound management via the addition of antimicrobial agents to the wound site. A number of dressings are used to clear the site of extraneous fluid/tissue either by absorption or using vacuum-assisted closure. A common disadvantage of dressings of this nature is the need to retain the dressing in position, which may not be possible depending on wound site.

There are a number of adverse effects associated with current regimes used in the management of wounds. These include protracted healing times, which may ultimately lead to the development of chronic wounds. Other undesirable effects relate to the qualities of the replacement tissues or organs that are generated via the healing process.

The absence of a universally accepted method for accelerating the healing of wounds is indicative of the need for novel medicaments and methods by which such acceleration may be effected. It is well recognised that there are failings and disadvantages associated with many of the current therapies available. Even in the case of relatively successful therapies, there is scope for improvement in terms of increased efficacy, or other parameters.

According to a first aspect of the invention there is provided the use of a compound that promotes oestrogenic activity in the manufacture of a medicament for the promotion of wound contraction. It will be appreciated that a medicament manufactured in accordance with this aspect of the invention should comprise an amount of the compound that promotes oestrogenic activity sufficient to promote wound contraction. Suitably the medicament may provide an amount of the substance that promotes oestrogenic activity sufficient to promote oestrogenic activity equivalent to that promoted by approximately 400 ng of 17β-oestradiol, per centimetre of wound.

In a second aspect, the invention provides the use of a compound that promotes oestrogenic activity in the manufacture of a medicament formulated to provide an amount of oestrogenic activity equivalent to that promoted by approximately 400 ng of 17β-oestradiol, per centimetre of a wound to which the medicament is administered.

In a third aspect of the invention there is provided a method of promoting wound contraction, the method comprising administering to a patient in need of such promoted wound contraction a therapeutically effective amount of a compound that promotes oestrogenic activity. The therapeutically effective amount of the compound that promotes oestrogenic activity may preferably be administered by means of a medicament in accordance with the present invention.

The present invention is based on the new and surprising finding that compounds that promote oestrogenic activity may be used to promote wound contraction. Wounds, the contraction of which has been promoted by means of the medicaments or methods of the invention, exhibit decreased wound area. This may be particularly apparent with respect to a reduced width of such wounds (wound width, for the purposes of the present disclosure, constituting a preferred indicator of wound area). This reduction in wound area both accelerates natural closure of the wound (i.e. closure without intervention), and facilitates the artificial closure of such treated wounds (for example closure by primary intention, using sutures or the like). In certain embodiments, the medicaments or methods of the invention are able to promote wound contraction without increasing the rate of re-epithelialisation occurring. The inventors have found that this profile of activity (promotion of wound contraction without promotion of re-epithelialisation) may be achieved using medicaments or methods of the invention in which oestrogenic activity per cm² of the body, or centimetre of wound, to which the medicament or method is provided is equivalent to that provided by 400 ng of 17β-oestradiol.

It had previously been recognised that oestrogenic compounds were capable of accelerating re-epithelialisation of wounds to which they were administered. However, prior to the present disclosure, it had not previously been recognised that they were capable of promoting wound contraction. The clinical contexts in which it would be wished to promote wound contraction may be very different to those in which it would be wished to accelerate the rate of re-epithelialisation. In many contexts where it may be wished to promote wound contraction, it would not generally be wished to accelerate the rate of re-epithelialisation, since to do so would provide no advantage, or may even prove disadvantageous.

By way of example, the inventors believe that the medicaments or methods of the invention may be used to promote contraction of large open wounds that are subsequently to be subject to grafting or suturing procedures. In these cases the ability to reduce wound area by promoting contraction will be advantageous as a “preparatory treatment” prior to grafting or suturing, since it will reduce the amount of graft material required, or the distance between wound edges to be sutured together. If re-epithelialisation of the wound bed occurs during such preparatory treatment it may interfere with the grafting healing process, and in particular may reduce integration of subsequently grafted material. Accordingly it will be seen that the ability of certain embodiments of the medicaments or methods of the invention to promote wound contraction without re-epithelialisation will be of particular benefit in the treatment of wounds prior to grafting or suturing.

Wound contraction is generally agreed to be dependent on the action of fibroblasts located both at the periphery of the wound and within the wound. Contraction is linked to fibroblast proliferation rate and connection of these cells to extracellular matrix components. Although they do not wish to be bound by any hypothesis, the inventors believe that therapeutically effective amounts of oestrogenic activity stimulate cytokine expression within the wound environment which increases fibroblast migration into the wound, and subsequent fibroblast differentiation and contraction.

The inventors believe that the wound contraction that may be promoted by the medicaments or methods of the invention may be promoted in wounds at any site in the body. However, it may be preferred that the wounds, contraction of which is to be promoted, are skin wounds.

Although the promotion of wound contraction may be of benefit in many clinical contexts, there will be certain wounds in which the promotion of contraction will not be beneficial. Typically such wounds may be wounds covering a relatively large surface area, or located over a joint or other articulation (where contraction may, in some circumstances, be associated with limitation of function). Examples of wounds for which it may be preferred not to promote contraction, using the medicaments or methods of the invention, include, but are not limited to, the group consisting of: graft donor sites; burns wounds; sunburn wounds; wounds associated with “skin peels” such as “chemical peels” (such as alphahydroxy acid peels, trichloroacetic acid peels or phenol peels) or laser peels; wounds associated with dermabrasion; wounds associated with dermaplaning; wounds associated with photorefractive keratectomy (PRK); and wounds associated with laser tattoo removal.

The medicaments or methods of the invention may be useful in promoting contraction of a wide range of wound types, including both acute wounds and chronic wounds. Examples of specific acute wounds and specific chronic wounds that may derive particular benefit from the medicaments and methods of the invention are considered elsewhere in the specification.

Various terms that are used in the present disclosure to describe the invention will now be explained further. The definitions provided below may be expanded on elsewhere in the specification as appropriate, and as the context requires.

### “Compound That Promotes Oestrogenic Activity”

The inventors have found that promotion of the contraction of wounds in accordance with the invention may be brought about using all compounds that promote oestrogenic activity tested to date.

The inventors believe that suitable compounds that promote oestrogenic activity for use in the medicaments or methods of the invention may be selected from the group consisting of: oestrogens; oestrogen receptor agonists such as ethinylyoestradiol, dienoestrol, mestranol, oestradiol, 17β-oestradiol, oestriol, conjugated oestrogens, piperazine oestrone sulphate, stilboestrol, fosfesterol tetrasodium, polyestradiol phosphate and tibolone; inhibitors of oestrogen or oestrogen receptor agonist breakdown; phytoestrogens; modulators of luteinising hormone; and chorionic gonadotrophin. As set out above, 17β-oestradiol constitutes a preferred compound that promotes oestrogenic activity to be used in the medicaments and methods of the invention.

### “Medicaments of the Invention”

For the purposes of the present disclosure, medicaments of the invention should be taken as encompassing any medicament manufactured in accordance with any aspect or embodiment of the invention. Suitable compositions, formulations and routes of delivery that may be used for medicaments of the invention are considered. Generally it will be preferred that medicaments of the invention will be topical medicaments.

It will be appreciated that the medicaments of the invention represent a preferred means by which the methods of the invention may be practiced.

### “Active Compound”

Except for where the context requires otherwise, for the purposes of the present disclosure, an “active compound” should be taken to be any compound that promotes oestrogenic activity, and hence contraction of wounds, in accordance with the present disclosure. Examples of suitable active compounds are provided elsewhere in the present disclosure, and favoured active compounds include 17β-oestradiol.

### “Therapeutically Effective Amount”

The term “therapeutically effective amount” as used in the context of the present disclosure when referring to a medicament of the invention, method of treatment of the invention, or an amount of an active compound (in accordance with the definition offered elsewhere) refers to an amount of the medicament, or of the method, or of an active compound, sufficient to provide oestrogenic activity sufficient to promote wound contraction. A therapeutically effective amount of medicament, method, or active compound should be sufficient to promote the contraction of a wound that has received the therapeutically effective amount.

As described elsewhere in the specification, the medicaments and methods of the invention may be used to treat existing wounds, or may be used prophylactically to treat sites at which a wound is to be formed, and thereby promote contraction of the wound once formed. It will be appreciated that in the event that treatment is of an existing wound, a therapeutically effective amount should be provided per centimetre of wound. In the event that prophylactic treatment is to be used, a therapeutically effective amount may be provided per cm² to which the prophylactic treatment is provided.

It may be preferred that a therapeutically effective amount of a compound that promotes oestrogenic activity is an amount of the compound that promotes oestrogenic activity equivalent to that promoted by approximately 400 ng of 17β-oestradiol, per centimetre of wound, or per cm² of the body to which the active compound is applied. Indeed, in the case that the active compound selected is 17β-oestradiol, a therapeutically effective amount may be approximately 400 ng.

### “Promotion of Oestrogenic Activity”

In the context of the present disclosure “promotion of oestrogenic activity” may be considered to encompass any promotion or increase in oestrogenic activity that is achieved on administration of an active compound. The oestrogenic activity to be promoted may preferably be the promotion of wound contraction, however other activities may also be investigated in the assessment of oestrogenic activity. It will immediately be appreciated that, once oestrogenic activity has been assessed, it is then a simple matter to determine whether or not such activity is, or has been, promoted on administration of a compound that may putatively have oestrogenic activity.

Oestrogenic activity may be assessed with reference to any one of a number of assays well known to the skilled person. Suitable assays include both in vivo and in vitro assays. For example, oestrogenic activity may be assessed in vivo by means of a rodent uterotrophic assay. Briefly, oestrogenic activity is demonstrated in such an assay by the ability of a compound to increase the weight of the uteruses of experimental rodents (such as rats). Compounds to be investigated for the ability to promote oestrogenic activity may be administered by dermal or oral routes.

Suitable in vitro assays for the assessment of oestrogenic activity may include the MCF-7 human breast cancer cell assay. In this assay oestrogenic activity of a compound is demonstrated by the ability of the compound to induce increased proliferation of the breast cancer cells.

In the case of either in vivo or in vitro assays, compounds having known oestrogenic activity, such as 17β-oestradiol, may be used as positive controls, and to produce dose response curves by which oestrogenic activity may be quantified. Such quantification may be particularly useful in assessing whether or not a compound of interest has oestrogenic activity making it suitable for use in accordance with the present invention.

### “Topical Medicament”

A “topical medicament”, for the purposes of the present disclosure, is to be construed as a medicament that is applied at a wound where it is intended to have its effect. Topical medicaments suitable for use in accordance with the present invention include, but are not limited to, ointments;.creams; lotions; gels; sprays; wound dressings capable of releasing active agents to the body; and injectable solutions administered by local injections (e.g. intradermal injections). Preferred routes of administration are those that allow the provision of a therapeutically effective amount of an active compound to a connective tissue (such as the dermis, in the case of skin wounds) surrounding or underlying a wound, the contraction of which is to be promoted. Topical medicaments represent preferred forms of the medicaments of the invention, and it is preferred that topical medicaments may be used to practice the methods of the invention.

Topical medicaments (i.e. those having their effect at the site, and preferably in the tissue, to which they are administered) will generally be preferred over medicaments or routes of administration associated with systemic administration of agents. For example, injectable medicaments of the invention may be for use in localised routes of administration, such as intradermal injection, rather than systemic routes of administration (such as intravenous, intraperitoneal, or subcutaneous injection).

### “Wounds”

For the purpose of the present disclosure wounds will primarily be described with reference to skin wounds, which comprise preferred wounds the contraction of which may be promoted in accordance with the present invention. However, the skilled person will appreciate that the promotion of wound contraction in accordance with the invention should preferably not be limited to skin wounds. The inventors believe that contraction of wounds may be promoted in wounds of all tissues, though these will preferably be of tissues other than those of the urinogenitary organs (here considered to comprise in particular the organs of the reproductive tract and the bladder). It is particularly preferred that the medicaments and methods of the invention be used to promote contraction of skin wounds.

Skin wounds, the healing of which may be accelerated using the medicaments and methods of the invention, include both chronic wounds and acute wounds. Examples of suitable chronic or acute wounds, the healing of which may be accelerated in accordance with the invention, are set out elsewhere in the specification.

Examples of specific wounds, other than those of the skin, which may benefit from accelerated healing of wounds in accordance with the present invention include, but are not limited to, those selected from the group consisting of: gastrointestinal ulcers, lung abscesses and wounds associated with myocardial infarction.

The inventors believe that medicaments or methods of the invention may be used to effectively promote the contraction of various gastrointestinal ulcers, such as peptic, gastric, duodenal and esophageal ulcers. These ulcers may be considered for the purposes of the present invention to constitute chronic wounds.

Lung abscesses may arise as a result of inflamed pleura, which lead to the formation of a pus-filled cavity and the subsequent loss of lung parenchyma. Presently, treatment of lung abscesses tends to involve the use of antibiotics to address the infection, but such treatment may still leave a hole in the lung that is eventually repaired to leave a dense scar. The presence of such a scar can significantly decrease lung function. The inventors believe that promotion of wound contraction using the medicaments and methods of the invention would be beneficial to speed up the repair process, and to decrease the size of the scar formed by decreasing wound size.

Myocardial infarction can result in coagulative necrosis, which in turn causes the activation of neutrophils to remove the necrotic debris. This process may result in the formation of a hole in the tissue that cannot be replaced by myocardium. The inventors believe that the promotion of wound contraction using the medicaments or methods of the invention may beneficially reduce the size of such a hole and thus aid suture techniques used in repair of the defect.

### “Treated Wounds”, “Control-treated Wounds” and “Untreated Wounds”

A “treated wound” in the context of the present disclosure is any wound that has been provided with a therapeutically effective amount of a medicament of the invention, or a therapeutically effective amount of a compound that promotes oestrogenic activity (such a compound may, for example, have been administered in accordance with a method of treatment of the invention).

“Control-treated wounds” and “untreated wounds” in the present context are respectively wounds treated with a relevant control, and wounds that have not been treated before, or during, healing. Control wounds will not be treated with a medicament of the invention, and preferably will not be treated with a therapeutically effective amount of an active compound. That said, wounds treated with medicaments known from the prior art may constitute suitable control wounds for comparative purposes (for example to illustrate increased efficiency or effectiveness of medicaments of the invention as compared to those already known). A “diluent control-treated wound” will be an untreated wound to which a control diluent has been administered, and a “naive control” will be an untreated wound made without administration of a compound that promotes oestrogenic activity, or a suitable control diluent, and left to heal without therapeutic intervention.

### “Centimetre of Wound”

A “centimetre of wound”, “wound centimetre” or “centimetre of wounding” in the context of the present disclosure constitutes a unit by which the size of a wound to be treated may be measured.

A wound centimetre may be taken to comprise any square centimetre of a body surface that is wounded in whole or in part. For example, a wound of two centimetres length and one centimetre width (i.e. with a total surface area of two centimetres²) will also be considered to constitute “two wound centimetres”, while a wound having a length of two centimetres and a width of two centimetres (i.e. a total surface area of four centimetres²) will constitute four wound centimetres. By the same token, a linear wound of two centimetres length, but of negligible width (i.e. with negligible surface area), will, for the purposes of the present invention, be considered to constitute “two wound centimetres”, if it passes through two square centimetres of the body surface.

The size of a wound in wound centimetres should generally be assessed when the wound is in its relaxed state (i.e. when the body site bearing the wounded area is in the position adopted when the body is at rest). In the case of skin wounds, the size of the wound should be assessed when the skin is not subject to external tension.

An inch of wound may be similarly defined, save that the relevant units of length or area are measured in inches rather than centimetres. The amount of an active compound that should be provided to promote healing of an inch of wound may be determined, based upon the information provided in the specification per centimetre of wounding, by use of a suitable conversion factor (one inch corresponding to approximately 2.54 centimetres).

A centimetre or inch of wounding may thus provide a unit by which the size of a wound to be treated may be measured, and the required amount of a medicament of the invention (or of an active compound administered in accordance with a method of treatment of the invention) may be determined.

### “Promoting Wound Contraction”

“Promoting wound contraction”, “promoting contraction of wounds”, or “promotion of wound contraction” in the context of the present disclosure should be taken to encompass any increase in the rate at which a wound contracts.

Promotion of contraction of a wound achieved using the medicaments or methods of the invention may preferably lead to a treated wound contracting at a rate at least 5% faster than an untreated or control wound, preferably at a rate at least 10% faster, more preferably at least 15%, 20% or 25% faster; yet more preferably at least 50% faster, still more preferably at least 75% faster, and most preferably 100% (or more) faster. Suitable methods by which promotion of wound contraction may be quantified to assess improvements in the rate of healing are described elsewhere in the specification. In general, suitable assessments of increases in wound contraction may be determined with reference to wound area (assessed microscopically or macroscopically, for instance by means of image analysis).

One measurement that may be used in assessing the rate of contraction of a wound is the rate at which the area of a wound decreases. Promotion of contraction of a wound achieved using the medicaments or methods of the invention may preferably lead to a treated wound in which wound area decreases at a rate at least 5% faster than an untreated wound, preferably at a rate at least 10% faster, more preferably at least 15%, 20% or 25% faster; yet more preferably at least 50% faster, still more preferably at least 75% faster, and most preferably 100% (or more) faster. The area of a wound may be assessed macroscopically or microscopically. The area of a wound may be determined directly on the wound in question, or by assessments of images (or other suitable representations) of the wound.

A preferred measurement that may be used in assessing the rate of contraction of a wound is the rate at which wound width decreases. Promotion of contraction of a wound achieved using the medicaments or methods of the invention may preferably lead to a treated wound in which wound width decreases at a rate at least 5% faster than an untreated wound, preferably at a rate at least 10% faster, more preferably at least 15%, 20% or 25% faster; yet more preferably at least 50% faster, still more preferably at least 75% faster, and most preferably 100% (or more) faster. Suitable methods by which wound width may be measured in order to assess promotion of contraction of wounds are described elsewhere in the specification. Preferably wound width may be measured microscopically, using histological sections, with reference to the distance between the wound margins (comprising the undamaged tissue defining the margins of the wounded area).

Promotion of contraction using the medicaments or methods of the invention may also give rise to a treated wound having an increased “healing age” when compared with an untreated or control treated wound. Such an increase in healing age may be assessed macroscopically, visually or clinically to determine maturity of the treated wound compared to a suitable untreated or control wound.

As noted elsewhere, the skin suffers from more direct, frequent, and damaging encounters with the external environment than any other organ in the body. As a result the skin suffers from more wounds than other organs, and it is therefore highly desirable to be able to promote the contraction of skin wounds in order that the wounds may be closed and this organ returned as rapidly as possible to its normal functional effectiveness.

Preferably, a wound the contraction of which is to be accelerated using the medicaments of the invention may be selected from the group consisting of: abrasions, cuts, pressure ulcers stages i) to iv); venous stasis ulcers, ulcers caused by mixed etiologies; lower extremity ulcers; diabetic ulcers; radiation ulcers; arterial ulcers; partial thickness excisions; full thickness excisions; wounds in the immunocompromised, elderly or paraplegics; pre-tibial lacerations; wounds that have been debrided; and surgical wounds (including surgical incisions or excisions).

It will be appreciated that promotion of contraction of wounds that may be achieved by the medicaments and methods of the invention may be of particular benefit in cases in which the wound healing response is impaired, inhibited, retarded or otherwise defective as compared to the normal rate of healing. The methods and medicaments of the invention may also be used to promote contraction of wounds in patients that are not subject to an impaired healing response. Illustrative examples of both contexts are set out below.

It is well known that dermal injuries in the aged heal more slowly than do those of younger individuals. The aged may therefore particularly benefit from promotion of wound contraction that may be brought about using the medicaments and methods of the invention. There are also many other conditions or disorders that are associated with a delayed or otherwise impaired wound healing response. For example patients with diabetes, patients with polypharmacy (for example as a result of old age), post-menopausal women, patients susceptible to pressure injuries (for example paraplegics), patients with venous disease, clinically obese patients, patients receiving chemotherapy, patients receiving radiotherapy, patients receiving steroid treatment or immuno-compromised patients may all suffer from impaired healing. In some cases the slower healing response exhibited by such patients may contribute to the development of infections at the site of wounds. The slow wound healing response may also be associated with the formation of chronic wounds, as considered below. Accordingly, it will be appreciated that such patients represent a preferred group that may benefit from increased wound contraction using the methods or medicaments of the invention.

The inventors believe that the medicaments and methods of the invention may be of particular value to aged or senescent patients. Aged or senescent patients, for the purposes of the present disclosure, may be defined as comprising patients aged 65 years or older, more preferably aged 75 years or older. In the case of female patients, it may be preferred that the medicaments or methods of the invention be provided to post-menopausal patients, to whom they may be of marked benefit.

Without detracting from the above, it may generally be preferred that the medicaments or methods of the invention may be utilised to promote contraction of wounds of patients not subject to delayed wound healing. Promoting contraction in this way will give rise to a faster wound healing response than would normally be achieved by such patients in the absence of promoted wound contraction (i.e. will give rise to faster healing than in control wounds). Accordingly the wounds of patients treated in this manner may be induced to heal more rapidly.

The skilled person will appreciate that there is a great benefit to be gained by society from the development of therapeutic agents and techniques that can hasten the healing of otherwise healthy patients. As well as the various benefits considered elsewhere in the specification, promotion of wound contraction in this manner can help reduce time spent in convalescence, and can thus benefit productivity. Accordingly, promotion of the contraction of wounds of healthy patients is a preferred embodiment of all aspects of the present invention.

The medicaments and methods of the invention may be used to promote the contraction of both chronic wounds and acute wounds. For the purposes of the present invention, a chronic wound may be defined as any wound that does not show any healing tendency within eight weeks of formation when subject to appropriate (conventional) therapeutic treatment. Acute wounds may be any wound other than a chronic wound.

Promotion of the contraction of chronic wounds is a preferred embodiment of the invention. Examples of chronic wounds that may benefit from promoted contraction provided by the medicaments or methods of the invention may be selected from the group comprising: leg ulcers; venous ulcers; diabetic ulcers; bed sores; decubitus ulcers; foot ulcers; and pressure ulcers. It will be appreciated that the long lasting nature of chronic wounds exacerbates many of the disadvantages associated with normal wound healing. For example, the duration of the period over which a patient suffering from a chronic wound will experience pain will generally be far longer than for a patient with an acute wound. Similarly the length of time over which desiccation as a result of liquid loss may occur will also be extended. Incidences of wound infection are also much increased in chronic, as opposed to acute, wounds. The ability of wound contraction promoted using the medicaments or methods of the invention to decrease the “open area” of treated wounds may be of benefit in reducing the possible ingress of pathogens, and also reducing fluid loss from the damaged tissue.

A preferred use of the medicaments or methods of the invention lies in their use to promote contraction of wounds prior to grafting or suturing to close the treated wound. This use is suitable for both chronic and acute wounds. In general the treatment of wounds using the medicaments or methods of the invention should be stopped prior to grafting or suturing, so that the grafted material, or sutured tissue, is not subject to further contraction once in situ.

Chronic wounds are also subject to many disadvantages that are not generally associated with acute wounds. For example, chronic wounds frequently expand beyond the limits of the original wounded area. This may arise as a result of infection (which may increase the damage around the margins of the wound, thereby leading to expansion) or through maceration of the tissue surrounding the wound (typically as a consequence of increased liquid loss through the chronic wound). The propensity for chronic wounds to expand beyond the boundary of the original injury means that such wounds are frequently of great surface area. The skilled person will appreciate that wound contraction promoted using the medicaments or methods of the invention may be useful in reducing the area of chronic wounds, and may help to prevent the expansion of such wounds.

Pretibial lacerations are acute wounds of the leg that are very frequently slow to heal, and which frequently give rise to the development of leg ulcers. Existing treatments used for pretibial lacerations include the use of surgical procedures (such as the use of skin grafts and flaps) in an attempt to heal the wound before chronic wound development. Pretibial, lacerations constitute acute wounds that may particularly benefit from treatment with the medicaments and methods of the invention, in order to promote contraction of the wound, thereby hastening healing and reducing incidences of chronic wound formation.

### Assessment of Promotion of Wound Contraction

Promotion of wound contraction may be demonstrated by an increase in the rate at which the area of a treated wound decreases. Such an increase in the rate at which the area of a wound decreases will indicate that the contraction of the wound in question has been promoted. The rate at which the area of a treated wound decreases may be compared with control wounds, or with reference data regarding the rate at which area of untreated wounds decreases in order to assess any difference in the rates observed.

The rate at which the area of a wound decreases provides an indication of the rate at which contraction of the wound is occurring. The increase in the rate of wound contraction that may be achieved using the medicaments or methods of the invention should be distinguished from the rate at which the wound is covered with a new epithelial layer (in the skin a new epidermis), which is related to the rate of re-epithelialisation. A preferred approach to measurement of the areas of experimental wounds may be to assess the width of such wounds histologically. The inventors have found that a decrease in the area of wounds will generally be observable with reference to a decrease in the width of such wounds. While the following paragraphs will generally refer to wound width, it will be appreciated that this measurement is representative of the area of a wound.

The skilled person will appreciate that increased wound contraction, leading to the formation of wounds that have a smaller area or narrower width, is important in the acceleration of healing, and provides the advantage that wound size is rapidly decreased, as is the “open” area of the wound. The decrease in wound width may be particularly beneficial in the context of wounds that are to be healed by primary intention, since the margins of the relatively narrow wounds may be readily apposed (and then held in apposition by sutures, or the like). Accordingly it will be appreciated that the use of the medicaments or methods of the invention to promote the contraction of wounds that are to be healed by primary intention represents a preferred embodiment of the invention.

The width of a wound (and hence the rate at which the width of a wound decreases, and so the promotion, or otherwise, of wound contraction) may be assessed macroscopically, or microscopically.

In the case of microscopic assessment of wound width, this may be undertaken using suitable histological slides. Preferably wound width may be measured at a standardised “reference” point within the wound. The inventors have found that measurements taken midway through the depth of the wound allow for an accurate and reproducible assessment of wound width. Suitable image analysis software may aid the assessment of wound width in this manner.

Macroscopic assessment of wound width may either be performed directly (i.e. with measurements taken directly from a wound), or indirectly, in which case measurements may be taken using representations of the wound, such as photographs, traced outlines, mouldings, or the like. Image analysis software may be useful in the macroscopic assessment of wound width, particularly as assessed from photographs. In assessing wound width macroscopically it is important that the width of a wound is differentiated from the degree of re-epithelialisation of the wound.

Whether assessed microscopically or macroscopically, the width (or area) of a wound should be determined with reference to the surrounding unwounded tissue, as opposed to epithelial coverage of the damaged site.

Given the potential for errors of measurement in macroscopic assessment of wound width it may generally be preferred to assess wound width microscopically rather than macroscopically.

### Preferred Routes of Administration and Suitable Formulations

Preferred routes of administration, by which therapeutically effective amounts of a compound that promotes oestrogenic activity may be administered to a wound the contraction of which it is desired to promote, are discussed more fully elsewhere in the specification. However, it may generally be preferred that compounds that promote oestrogenic activity are provided by local administration to the wound, contraction of which is to be promoted. Suitable methods by which such local administration may be achieved will depend on the identity of the tissue in question, and may also be influenced by the size or location of the wound. Preferred routes of administration may include local injection (for example intradermal injection in the case where it is wished to promote contraction of wounds of the skin). Other suitable means of administration include the use of topical medicaments such as sprays; powders; drops (e.g. for the ear); ointments or creams; or release from local devices e.g. stents, implants, polymers, wound dressings, or the like. Examples of solid or liquid medicaments of the invention, and suitable formulations that may be used, are considered elsewhere in the specification.

Generally, medicaments of the invention may be formulated and manufactured in any form that allows for the medicament to be administered to a patient such that a therapeutically effective amount of the compound that promotes oestrogenic activity is provided to a wound the contraction of which is to be promoted. In particular, the inventors believe that preferred routes of administration should be capable of delivering an active compound to a connective tissue underlying or surrounding a wound (or a site where a wound is to be formed).

It will be appreciated that certain routes of administration normally associated with systemic administration may also be suitable for topical administration of active compounds to an otherwise “inaccessible” wound in which it is desired to promote contraction (for example, inhalation or intranasal administration of compounds that promote oestrogenic activity may be of use in promoting contraction of wounds of the respiratory system).

Medicaments of the invention may preferably be provided in the form of one of more dosage units providing a therapeutically effective amount (or a known fraction or multiple of a therapeutically effective amount) of a compound that promotes oestrogenic activity. Methods of preparing such dosage units will be well known to the skilled person; for example see Remington's Pharmaceutical Sciences 18^{th} Ed. (1990).

The medicaments of the invention should be taken to encompass any composition, material or device from which a compound that promotes oestrogenic activity may be provided to a wound in a therapeutically effective quantity (as defined elsewhere in the specification).

Examples of suitable compositions that may be utilised as medicaments of the invention include: creams, emulsions, ointments, irrigation solutions, sprays, foams, powders, gels, wound dressings, microneedles, liposomes, nanomicelles, thermosetting gels, microparticles, nanoparticles, crystals, biomaterials, stents, films and products suitable for use as artificial skin.

Medicaments of the invention, such as irrigation fluids of the invention, may additionally comprise one or more agents independently selected from the group consisting of: cleansers; antibiotics; antifungal agents; antiseptic agents; and anaesthetic agents. Medicaments of this sort may be of particular value in the treatment of cavitating and/or chronic wounds.

Examples of suitable materials that may be used as medicaments of the invention include: fabrics (such as woven and non-woven materials) including bandages, sticking plasters, patches, swabs, or other wound dressings.

Examples of wound dressing materials that may be used in the production of medicaments in accordance with the present invention may include dressings in the following broad classes.

“Passive products”, comprising traditional dressings that provide cover over the wound (such as gauze and tulle dressings)

“Interactive products” comprising polymeric films and forms which are mostly transparent, permeable to water vapour and oxygen, non-permeable to bacteria (hyaluronic acid, hydrogels, foam dressings)

“Bioactive products” comprising dressings that are particularly suitable for the delivery of substances active in wound healing, such as compounds that promote oestrogenic activity. Examples of bioactive products include hydrocolloids, alginates, collagens and chitosan, and these constitute preferred examples of materials that may be used in the medicaments of the invention.

Suitable examples of wound dressings that may be used in the medicaments of the invention may be selected from the following:

### “Gauze Dressings”

Gauze dressings can stick to the wound surface and disrupt the wound bed when removed. As a result gauze dressings will generally only be used on minor wounds or as secondary dressings.

### “Tulle Dressings”

Tulle dressings do not stick to wound surfaces. They are suitable for use in flat, shallow wounds, and are useful in patients with sensitive skin. Examples of tulle dressings known from the prior art include Jelonet® and Paranet®.

### “Film Dressings” and “Semipermeable Film Dressings”

Film dressings and semipermeable film dressings comprise sheets of materials that may be used to cover wounds. Such dressings may preferably comprise sterile materials. Suitable materials from which such films may be manufactured include polyurethane and chitin. Film dressing (or semipermeable film dressings) may be coated with adhesives, such as acrylic adhesives, in order to assist their retention at sites where they are required. Dressings of this type may be transparent, and therefore allow the progress of wound healing to be checked. These dressings are generally suitable for shallow wounds with low exudate. Examples known from the prior art include OpSite®, Tegaderm®

### “Hydrocolloid Dressings”

Hydrocolloid dressings may comprise carboxymethylcellulose, gelatin, pectin, elastomers and adhesives that turn into a gel when exudate is absorbed. This creates a warm, moist environment that promotes debridement and healing. Depending on the hydrocolloid dressing chosen they may be suitable for use in wounds with light to heavy exudate, sloughing or granulating wounds. Dressings of this sort are available in many forms (adhesive or non-adhesive pad, paste, powder) but most commonly as self-adhesive pads. Examples known from the prior art include DuoDERM®, Tegasorb®

### “Hydrogel Dressings”

Hydrogel dressings are composed mainly of water in a complex network or fibres that keep the polymer gel intact. Water is released to keep the wound moist. These dressings may be used for necrotic or sloughy wound beds to rehydrate and remove dead tissue. They should not be used for moderate to heavily exudating wounds. Examples known from the prior art include Tegagel®, Intrasite®

### “Alginate Dressings”

Alginate dressings are composed of calcium alginate (a seaweed component). When in contact with wound, calcium in the dressing is exchanged with sodium from wound fluid and this turns the dressing into a gel that maintains a moist wound environment. These dressings are good for exudating wounds and help in debridement of sloughing wounds. In general they should not be used on low exudating wounds as this will cause dryness and scabbing. Alginate dressing should be changed daily. Examples known from the prior art include Kaltostat®, Sorbsan®

### “Polyurethane or Silicone Foam Dressings”

These dressings are designed to absorb large amounts of exudates. They maintain a moist wound environment but are not as useful as alginates or hydrocolloids for debridement. In general they should not be used on low exudating wounds as this will cause dryness and scabbing. Examples known from the prior art include Allevyn®, Lyofoam®

### “Collagen Dressings”

Collagen dressings are generally provided in the form of pads, gels or particles. They promote the deposit of newly formed collagen in the wound bed, and absorb exudate and provide a moist environment.

Suitable dressings that may be used as, or in, solid medicaments in accordance with the invention include foam or sponge dressings (i.e. foams in which the matrices in which the pores are located are solid, as opposed to liquid foams considered elsewhere in the disclosure). Such foam or sponge dressings should be capable of providing a therapeutically effective amount of a compound that promotes oestrogenic activity to a wound to which the dressing is applied. Many materials from which suitable foam dressings may be manufactured are known to those skilled in the art, and these include polyvinyl alcohol (PVA) and other similar materials.

Compounds capable of promoting oestrogenic activity may be incorporated in, or applied to, foams or sponges for use in wound dressings by any suitable technique known in the art. For example, suitable foams or sponges may be coated with compounds that promote oestrogenic activity, or impregnated with such compounds. It will be appreciated that the important concern is merely that the oestrogenic compound be incorporated in, or applied to, the foam or sponge in such a manner that a therapeutically effective amount of the compound that promotes oestrogenic activity to a treated wound.

The skilled person will be aware that a suitable wound healing dressing to be used on a particular wound may be selected with reference to the type of the wound, size of the wound, and healing progression of the wound. Generally, the selection of an appropriate wound dressing may be determined with reference to Table 1.

TABLE 1

| Wound type | Dressing type |
|---|---|
| Clean, medium-to-high | Paraffin gauze |
| exudate (epithelialising) | Knitted varicose primary dressing |
| Clean, dry, low exudate | Absorbent perforated plastic film-faced |
| (epithelialising) | dressing |
| | Vapour-permeable adhesive film dressing |
| Clean, exudating | Hydrocolloids |
| (granulating) | Foams |
| | Alginates |
| Slough-covered | Hydrocolloids |
| | Hydrogels |
| Dry, necrotic | Hydrocolloids |
| | Hydrogels |

The medicaments or methods of the invention may make use of a number of different types of wound dressings in which compounds that promote oestrogenic activity are incorporated in a dressing material such that a therapeutically effective amount of the compound that promotes oestrogenic activity is releasable to a wound.

Suitable examples may include: nonresorbable gauze/sponge dressing for external use (typically a sterile or nonsterile device intended for medical purposes, they may be placed directly on a patient's wound to absorb exudate, and are generally made from open woven or nonwoven mesh cotton cellulose or a simple chemical derivative of cellulose); hydrophilic wound dressing (again, may be sterile or nonsterile in form, are intended to cover a wound and to absorb exudate, and are manufactured of nonresorbable materials such as cotton, cotton derivatives, alginates, dextran, and rayon that have hydrophilic properties and are capable of absorbing exudate); occlusive wound dressing (which are nonresorbable, sterile or nonsterile device intended to cover a wound and thus provide or support a moist wound environment, while allowing the exchange of gases such as oxygen and water vapor through the device, and which tend to be manufactured from synthetic polymeric materials, such as polyurethane, with or without an adhesive backing); and hydrogel wound and burn dressing (available in sterile or nonsterile forms that are used to cover a wound, and thereby to absorb wound exudate, to control bleeding or fluid loss, and to protect against abrasion, friction, desiccation, and contamination; they tend to be manufactured from nonresorbable matrices, such as those made of hydrophilic polymers or other material in combination with at least 50% water). It will be appreciated that the incorporation of compounds that promote oestrogenic activity in such dressings will generally lead to their classification as “interactive dressings” under present FDA guidelines.

In the case of a medicament of the invention comprising a solid material it may be preferred that the medicament be formulated such that a predetermined area of the medicament provides a therapeutically effective amount of a compound that promotes oestrogenic activity to a wound to which the solid material is applied.

By way of example, a solid medicament may be formulated such that each square centimetre of the medicament provides an amount of oestrogenic activity equivalent to that produced by approximately 400 ng of 17β-oestradiol. It will be appreciated that, in the case that it is desired to administer 17β-oestradiol as the active compound, such a medicament may, therefore, be formulated such that it provides approximately 400 ng of 17β-oestradiol per square centimetre of the medicament.

In the case of a liquid medicament it may be preferred that the medicament is administered in sufficient quantity to provide the specified therapeutically effective amount of oestrogenic activity.

It may be preferred that medicaments of the invention are formulated to provide discrete dosage units capable of providing a specified amount of oestrogenic activity (this specified amount of oestrogenic activity will generally be a known fraction or multiple of a therapeutically effective amount of the oestrogenic activity promoted by a chosen compound). For instance dosage units may be formulated with reference to the size of a wound to be treated. It may be envisioned that medicaments in accordance with this embodiment of the invention will be formulated so that a specified quantity (for instance a specified volume or a specified weight) of the medicament may be administered to a specified size of wound (for instance a specified area of wound or a specified length of wound) in order that a therapeutically effective amount of an active compound may be administered to the wound. For instance, a suitable medicament may be formulated so that a specified quantity of the medicament (for example 0.1 nL to 100 mL of the medicament, or 0.1 ng to 100 grams of the medicament) provides an amount of oestrogenic activity equivalent to that produced by approximately 400 ng of 17β-oestradiol. It may be preferred that the specified quantity of the medicament be provided per centimetre of wounding.

Suitable specified quantities of medicaments of the invention may be selected with respect to the nature of the medicament in question. By way of example, it may be preferred that the quantity of an injectable solution medicament of the invention required to administer a therapeutically effective amount of an active compound may relatively small (for example in the region of 0.05 to 0.5 ml). In contrast, the quantity of a gel, ointment or spray medicament of the invention required to administer a therapeutically effective amount of an active compound may relatively larger (for example in the region of 0.25 to 2.5 ml). The quantity of an irrigation fluid medicament of the invention required to administer a therapeutically effective amount of an active compound may larger still (for example in the region of 0.5 to 5 ml, or more).

It will be appreciated that the concentration of active compounds within the different forms of the medicaments of the invention may be selected, for example in accordance with the volumes outlined above, in order to provide suitable medicaments of the invention capable of providing therapeutically effective amounts of an active compound. Thus, in the case that it is wished to deliver a therapeutically effective amount of approximately 400 ng of 17β-oestradiol, this amount of the active compound may be incorporated in a volume of between 0.05 and 0.5 ml of an injectable solution, in a volume of between 0.25 and 2.5 ml of a gel, ointment or spray, and in a volume of between 0.5 and 5 ml, or greater, of an irrigation fluid. Suitable concentrations of other active compounds that may be used in the medicaments of the invention will be readily apparent to those skilled in the art.

Medicaments of the invention may be provided in the form of discrete dosage units capable of providing a therapeutically effective amount of an active compound (which is to say capable of providing an amount of a medicament sufficient to provide a therapeutically effective amount of oestrogenic activity). A suitable dosage unit in accordance with this embodiment of the invention may comprise a sufficient amount of a medicament of the invention to promote the contraction of a given length or area of a wound.

A suitable dosage unit may comprise sufficient of a medicament of the invention to promote the contraction of one centimetre of a wound. Alternatively a suitable dosage unit may comprise sufficient of a medicament of the invention to promote the contraction of one inch of a wound. It will be appreciated that medicaments of the invention may be formulated to provide single dosage units or to provide multiple dosage units, as required. Thus a medicament of the invention may be packaged to provide one or more dosage units. Each dosage unit may provide a known fraction or multiple of a therapeutically effective amount of a compound that promotes oestrogenic activity.

Suitable forms in which such discrete dosage units may be provided can be selected with reference to the nature of the medicament to be administered. Merely by way of example, medicaments of the invention comprising injectable solutions may be provided in the form of vials or pre-filled syringes comprising one or more dosage units. Other liquid medicaments in accordance with the invention, such as gels, creams, ointments, irrigation fluids or the like, may be provided in the form of tubes, sachets, cartons or blister packs comprising one or more dosage units.

Solid medicaments of the invention may readily be formulated such that a given area of the solid medicament is capable of providing sufficient of an active compound (i.e. sufficient therapeutically effective oestrogenic activity) to promote contraction of a matching-sized area of wound. In such an embodiment a solid medicament of the invention may be cut to the required size and/or shape to cover a wound, and the medicament will release a therapeutically effective amount of an active compound sufficient to promote the contraction of the wound. In particular, such a solid medicament may provide oestrogenic activity equivalent to that provided by 400 ng of 17β-oestradiol to each cm² to which the medicament is provided.

Furthermore, since it will be appreciated that the therapeutically effective amounts of oestrogenic activity provided by the medicaments of the invention are so low that they will generally not be detrimental to unwounded tissue, the skilled person will appreciate that a solid medicament, formulated such that a given area of the medicament can provide sufficient oestrogenic activity to promote contraction of a corresponding area of wound, may be placed over an area that includes both wounded and unwounded tissue without the need to shape the medicament such that it conforms to the wounded area alone. Contact between the medicament and the wounded area covered will ensure that a therapeutically effective amount of an active compound will be provided to the wound, thereby promoting its contraction, while the amount of oestrogenic activity provided to the unwounded area will generally be insufficient to induce adverse effects. Indeed, in the case of organs, such as the skin, where the unwounded organ is relatively impermeable to compounds that promote oestrogenic activity, very little of the active compound may actually be able to enter the unwounded portion.

The period of time over which this therapeutically effective amount of a compound that promotes oestrogenic activity is administered may be selected with reference to the nature of the medicament. For example medicaments intended to remain in contact with the wound for a protracted period of time (such as bandages, sticking plasters, or other dressings) may be formulated such that the therapeutically effective amount of a compound promoting oestrogenic activity is administered relatively slowly. In contrast, medicaments that are intended only to be placed briefly in contact with the wound (such as swabs that will generally be used to “wipe down” a wound) may be formulated such that the therapeutically effective amount of the compound that promotes oestrogenic activity is administered in the relatively short time in which the medicament is in contact with the wound.

Suitable formulations may be selected with reference to how readily the compound that promotes oestrogenic activity is liberated from the solid medicament. For instance, in the case of a bandage, or the like, in which it is desired that release of the compound that promotes oestrogenic activity may be relatively slow, the compound that promotes oestrogenic activity may be incorporated in a matrix in which the interstices are sized such that the compound is released slowly to the wound. Alternatively, or additionally, the compound that promotes oestrogenic activity may be incorporated in a formulation from which it will be slowly released into the wound (in response to wound moisture, acidity, enzyme activity, or the like), as considered elsewhere in the specification. By contrast, in the case of a swab, or the like, in which it is desired that release of the active compound is relatively rapid, it may be desired that the compound which promotes oestrogenic activity be provided in a liquid carrier that may be released rapidly from the substrate thereby providing the compound rapidly to the wound. In such uses the compound that promotes oestrogenic activity may be formulated in such a way that it is readily “accessible” to the wound (for example, dissolved in a suitable solute without the presence of binding partners or other agents that will form complexes with the oestrogenic compound).

Compounds that promote oestrogenic activity for use in medicaments of the invention may be provided as solid or liquid formulations. In either solid or liquid formulations the active compound will be incorporated in a carrier, from which the active compound will be released to a wound in order to exert its therapeutic activity.

Solid formulations for use in medicaments of the invention may include excipients such as binders. Suitable binders will be well known to those skilled in the art.

Solid formulations for use in medicaments of the invention may, or may not, be contained in a containment membrane or coating, microspheres, microgranules or microcapsules. The materials for such containment membranes or coatings may be selected from any of a variety of biodegradable natural or synthetic materials. Suitable materials may provide resistance to diffusion of the active compound.

It may be preferred that suitable materials for use in containment membranes or coatings be selected such that they allow sustained release of the active compound to the wound. Examples of techniques by which this may be achieved will be well known to those skilled in the art, and will include the use of alternating layers of a suitable containment membrane or coating with layers of a carrier incorporating the active compound.

Suitable materials for use in containment membranes or coatings will generally degrade or be broken down over a period of time, thereby exposing the carrier, and allowing therapeutic release of the active compound from the carrier to the wound. The degradation or breakdown of suitable containment membranes or coatings may be caused by prolonged exposure to a wound. Factors that may mediate the degradation of such containment membranes or coatings will generally be the same as those that may cause the release of the active compound from the carrier. These are considered in more detail below, but it will be appreciated that the degradation or breakdown of containment membranes or coatings may typically be caused by moisture associated with the wound, or by the activity of enzymes active during wound healing.

Suitable solid formulations that may be used in medicaments of the invention may, for example, be selected from: powders; sprays, crystals; microneedles; solid compositions comprising microparticles, nanoparticles or liposomes; biomaterials; stents; films; artificial skin substances and wound dressings.

Suitable liquid formulations that may be used in medicaments of the invention may, for example, be selected from: gels: thermosetting gels; creams; ointments; sprays; injectable solutions; irrigation solutions; other solutions of oestrogenic compounds; and liquid compositions comprising microparticles, nanoparticles or liposomes.

In the case where the medicament of the invention is a wound dressing, suitable solid formulations may be applied throughout the dressing, and particularly to the surface of the dressing that is to be placed into contact with the wound. Solid formulations may be applied as a coating that may be applied to all the material of the dressing, or may be applied to discrete portions of the dressing (for instance the surface of the dressing that is to be placed in contact with the wound). Solid formulations may also be provided as granules, microgranules, microparticles, nanoparticles or liposomes adhered to the material of the dressing.

It will be appreciated that one of the advantages of applying solid formulations incorporating compounds that promote oestrogenic activity to wound dressings in this manner is that the release of the active compound to the wound, in order to provide a therapeutic amount of the active compound, occurs from the carrier, rather than from the material of the wound dressing itself. Thus the release characteristics of the carrier may be selected to achieve the optimal therapeutic effect of the active compound, while the physical properties of the material making up the wound dressing can be independently selected with a view to providing maximum effectiveness in term of compression, absorbency, or the like. In such cases it may generally be preferred that the material making up the wound dressing be one that is relatively “inert”, that is to say a material that does not in itself exert an effect on cells in the wound (save for any effects that may arise as a result of the material's structure, it's ability to absorb liquid from the wound, it's ability to exert compression on the wound, or the like).

Thus, merely by way of example, a sponge dressing material having pore/cell size that is well suited to drawing excess exudate from a wound, but which is of a material that is not well adapted for the release of active compounds, may be provided with a coating of a solid formulation that incorporates an active compound in a carrier that has characteristics optimised for the release of therapeutic amounts of the compound. In this way the beneficial effects of the material of the dressing may be combined with the beneficial characteristics of the chosen carrier.

The pore sizes of foams or sponges for use in medicaments of the invention may be such that they prevent substantial ingress of cells contributing to the wound healing process (e.g. keratinocytes associated with re-epithelialisation, or cells involved in granulation tissue formation) into the dressing. Foams or sponges to be used in medicaments of the invention may also be subject to surface treatments designed to reduce their adhesion to wounds to which they are applied. This may be advantageous in reducing damage that may otherwise be associated with changing dressings applied to wounds.

Foam or sponge pore sizes may be selected such that they facilitate the removal of exudates from the wound area, thus preventing or reducing maceration of the wound. The removal of exudates may be facilitated by the application of negative pressure to treated wounds, or to wound dressings applied to such wounds, and this may have further beneficial effects on the healing response.

Typically, release of the active compound to a wound may be brought about by the dissolution of the carrier in which the active compound is incorporated in moisture associated with (e.g. released from) the wound. In such cases it will be appreciated that suitable carriers may include water soluble compositions.

Other approaches may also be taken to control the release of therapeutic amounts of an active compound to a wound. For example, release of the active compound may be mediated by enzyme activity present in the wound. It is known that various proteloytic enzymes, such as plasmin and the Matrix Metalloproteinases (MMPs) are released during the process of wound healing. The natural proteloytic activity of such enzymes may be used to control release of the active compound to the wound. In this case, the carrier in which the active compound is incorporated may be one which is subject to proteloytic degradation by enzymes released during the wound healing response. For example, in the case of release mediated by the proteloytic enzyme MMP-1, the active compound may be incorporated in a carrier comprising a collagen matrix, wherein degradation of the collagen by MMP-1 will cause release of the active compound. Other examples of genes encoding proteolytic enzymes whose expression is significantly upregulated one day post-wounding, and which may therefore be used to control release of active compound into a wound where contraction is to be promoted, include cathepsin C, cathepsin L and MMP-9.

It has been suggested that the pH of wounds is lower than that of unwounded skin, and this observation may be considered when devising medicaments of the invention. Accordingly, carriers suitable for use in medicaments of the present invention may be ones in which the decreased pH of the wound to be treated contributes to therapeutic release of the active compound. Suitable materials that may be used in carriers in accordance with this embodiment of the invention will be well known to those skilled in the art.

An example of a pH-controlled carrier that releases a bound active compound in response to low pH can be found in a review by Gillies et al. (Pure Appl. Chem., 2004, 76, 1295-1307). Bae and co-workers (J. Controlled Release, 2003, 90, 363-374; ibid. 2003, 91, 103-113) prepared pH-sensitive micelles from poly(L-histidine)-block-PEO[PEO-b-P(His)], shown below. The micelles were prepared at pH 8, but were destabilised below pH 7.4 as evidenced by light transmittance measurements, light scattering, and fluorescent probe techniques. The triggering pH could be adjusted within the range of 7.2-6.6 by incorporation of different amounts of poly(L-lactic acid)-block-PEO(PLLA-b-PEO). Release of an active compound (in this experimental case the anti-cancer drug doxorubicin) could be modulated using pH, and it was found that in vitro activity of the doxorubicin-loaded mixed micelles was heavily dependent on concentration.

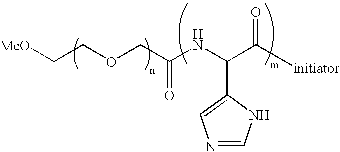

### Poly(L-histidine)-block-poly(ethelene oxide)

Although the passages above consider the benefits that may be provided when using different substances as a wound dressing material and as a carrier for the active compound, it will be appreciated that there may be circumstances in which it is desired that the material of a wound dressing serve as the carrier.

The use, in a medicament, of the same substance as both the material of a wound dressing and also as a carrier for an active compound may provide advantages in terms of reduced costs in the manufacture of such a medicament. It will be appreciated that medicaments in accordance with this embodiment of the invention may either be manufactured directly from the selected material already comprising the active compound, or may be manufactured before being treated such that they contain the active compound.

An example of the former suggestion may be the case in which a material (such as a hydrogel, collagen gel or other suitable polymer) containing an active agent in accordance with the invention is shaped or otherwise treated to provide a wound dressing (or part thereof). An example of the latter suggestion may be the case in which a material (such as an absorbent wound dressing) is treated with a solution containing the active compound, and the solution allowed to at least partially evaporate, thereby leaving a substantially dry wound dressing permeated with the active compound. The active compound may then be released from the dressing material by dissolution in fluids released from the wound.

Medicaments of the invention may be formulated in the form of compositions comprising the active compound, which promotes oestrogenic activity, in a polymer matrix. Suitable polymer matrices may typically comprise materials selected from the group consisting of: acrylic copolymers; vinyl acetate; natural or synthetic rubbers;

The skilled person will be readily able to identify polymer compositions that may be used in the manufacture of medicaments of the invention from those known in the prior art. Specific examples of formulations comprising polymer matrices suitable for use in medicaments of the present invention are described in greater detail below.

A suitable matrix that may be used for transdermal delivery of active compounds in medicaments of the present invention is described in U.S. Pat. No. 5,252,334 and in U.S. Pat. No. 5,770,219. The disclosure of these documents, particularly in their description of the manufacture of such matrices, is incorporated by reference.

The matrix described in these documents is formed of a skin-adhesive acrylate copolymer, and attains rates of delivery of the active compound suitable to provide therapeutically effective amounts thereof to a wound. For example, the matrices described in U.S. Pat. No. 5,252,334 can be used to administer approximately 0.2 μg of 17β-oestradiol per hour per square centimetre of skin. This rate of delivery described may be achieved without the addition of drug delivery rate enhancers. The skilled person will immediately appreciate that increased rates of administration of an active compound, for example to provide a dose in the region of 0.4 μg of 17β-oestradiol per centimetre of skin over a 24 to 48 hour period, may be readily obtained by proportionately increasing the amount of the active compound incorporated in the matrix.

Alternatively or additionally, the matrices and formulations described in U.S. Pat. No. 5,252,334 may be applied to a wound for a period of time sufficient to allow a therapeutically effective amount of an active compound to be administered to a wound (or a site where a wound is to be formed). It will be appreciated that a suitable period of administration may be in the region of an hour or less (for example half an hour), and that exposure to a medicament of the invention for such relatively short periods of time may be undertaken as part of the immediate care (such as post-operative care) associated with surgical interventions. The relatively short time in which medicaments in accordance with this embodiment of the invention may release a therapeutically effective amount of an active compound to a wound, or a site where a wound is to be formed, is particularly advantageous in the context of surgical procedures since the medicament need only be applied relatively briefly (for example in the time during which a patient is being prepared for surgery) to allow therapeutically effective wound contraction to be effected.

A suitable medicament comprising an active compound incorporated in a matrix of the type described in U.S. Pat. No. 5,252,334 may consist of a first layer laminated to a second layer, where the first layer effective acts as a backing sheet (made of a material which is substantially impermeable to the active compound) and the second layer comprises an adhesive matrix in which the active compound is incorporated (the adhesive matrix comprising a copolymer of 2-ethylhexyl acrylate and at least one co-monomer selected from the group consisting of vinyl acetate, acrylic acid, and methyl acrylate). The adhesive matrix thus secures the medicament to the site to be treated, and also administers a therapeutically effective amount of the active compound. The backing sheet can function to protect the matrix containing the active compound, as well as shielding external materials from the adhesive layer, and preventing unwanted release of the active compound to tissues other than those to be treated.

U.S. Pat. No. 4,814,168 describes “dermal compositions” that may be used as a suitable matrix by which active compounds of the invention may be administered.

The compositions of U.S. Pat. No. 4,814,168 comprise an active ingredient incorporated in a multi-polymer of vinyl acetate, polyethylene and optionally one or more monomers, a natural or synthetic rubber and a tackifying agent. The ratio of the multipolymer to the rubber is, respectively, about 1:1 to about 10:1, although this may more preferably be between 1:1 and 5:1, and even more preferably 3:1. The multi-polymer can be a copolymer, or terpolymer also including an acrylic and/or methacrylic acid monomer. The composition can additionally contain or employ other ingredients known for use in pressure sensitive adhesives including crosslinking agents, plasticizers, fillers and anti-oxidants. Dermal compositions of this type suitable for use as matrices from which therapeutically effective amounts of active compounds may be administered in accordance with this embodiment of the invention can optionally contain a crosslinking agent, tackifiers, penetration enhancers and other ingredients known for use in adhesives for the transdermal delivery of drugs.

The dermal compositions can be produced by a variety of methods known to those skilled in the art. By way of example, these may include the homogenous mixing of the active compound multi-polymer, and optional crosslinking agent and additional ingredients in aqueous solution followed by removal of excess water. The composition is prepared by mixing the active compound and an essentially non-tacky polymer, (namely the multi-polymer) with an elastomer (namely the rubber) and a tackifying agent. The composition maintains its adhesive properties even where the active acts as a plasticizer or solvent. The tackifying agent increases tack and adhesiveness.

Although the structure of the compositions described in U.S. Pat. No. 4,814,168 has not been analyzed, it is suggested that the two polymers incorporated in the compositions result in a heterogeneous mix, the elastomer performing as an interpenetrating polymeric network in the multi-polymer.

A further example of a suitable formulation by which active compounds may be administered to a wound (or site to be wounded) comprises micellar nanoparticles incorporating the active agent. Suitable micellar nanoparticles may be made by hydrating a mixture of an oil, a stabilizer/surfactant, and an alcoholic initiator (normally ethanol or methanol) with an aqueous solution comprising the active compound. A method by which micellar nanoparticles may be produced is described fully in U.S. Pat. No. 5,629,021. The methods of manufacturing micellar nanoparticles comprising active compounds described in that disclosure are incorporated by reference. The micellar nanoparticles that may be produced using the methods described in U.S. Pat. No. 5,629,021 are normally less than 100 nanometres in diameter.

The use of micellar nanoparticles confers a number of advantages in the context of the present invention. One specific advantage of such materials lies in their delivery of natural or synthetic hormones, such as 17β-oestradiol, that will frequently be used as active compounds in the medicaments or methods of the invention. These materials often have solubility problems in that they are often only soluble in materials such as ethanol. It is known that ethanolic solutions can be difficult to incorporate in stable particulate systems. The inventors have found that ethanol represents a preferred solvent for use in the medicaments of the invention. Micellar nanoparticles offer a notable advantage in that they allow materials that are soluble in any solute selected from water, oil, or an initiator (i.e., ethanol or methanol) to be incorporated into stable particles with mean diameters between about 30 and 1000 nanometres.

The small size of micellar nanoparticles marks them apart from many other compositions that may be used for the administration of therapeutic amounts of compounds that promote oestrogenic activity, and also lends itself to certain of their applications. The skilled person will recognise that other synthetic particles (such as liposomes, nonphospholipid lipid vesicles and microcapsules) are normally a micron, or larger, in size. In contrast, micellar nanoparticles may have mean diameters between about 30 and 1000 nanometres, and will frequently have sizes less than 100 nanometres diameter. Most preparations have particle diameters between 30 to 500 nanometres, are mixable in water, and filterable through either 0.2 or 0.45 micron filters.

Another notable advantage of micellar nanoparticles lies in their improved delivery of active compounds (such as 17β-oestradiol) to a wound. Delivery of the active compound, even through intact skin (as in cases of prophylactic treatment prior to wounding) is facilitated, since the small size of the micellar nanoparticles allows their easy penetration through the epidermis and into the dermis. Micellar nanoparticles comprising 17β-oestradiol have been shown to be effective in the topical administration of that compound to the skin.

Finally, micellar nanoparticles can be stored stably for protracted periods at temperatures of between −20 and 25° C. This provides clear advantages in cases where it is wished to produce medicaments of the invention that will have relatively long “shelf-lives” between manufacture and use.

Although the preceding paragraphs generally consider provision of medicaments to existing wounds it will be appreciated that these are also generally suitable for administration or application to sites where a wound is to be formed, in order to prophylactically induce contraction of the wound when formed.

It is known that certain agents can increase the ability of active compounds of the invention to cross the skin. These agents are typically referred to as “permeation enhancers”, terminology that will be adopted in the present disclosure.

The need for permeation enhancers may arise since the intact skin provides an effective barrier to the uptake of active compounds that may be used in accordance with the invention. Thus it will be appreciated that permeation enhancers are most likely to be of benefit in medicaments or methods that are to be utilised prophylactically (discussed elsewhere, in which medicaments or methods of the invention are administered prior to the formation of a wound in order to “prime” the treated area so a wound formed at that site will benefit from the promotion of wound contraction as soon as the wound is formed), since in these cases the active compounds of the medicaments will have to pass through the unwounded skin in order to reach the underlying connective tissue, where the inventors believe that their therapeutic effects are mediated.

In the case of medicaments administered to sites of existing wounds, the wound will generally impair the skin's barrier function. As a result, this will normally facilitate the uptake of therapeutically effective amounts of the active compounds from medicaments of the invention without the need for permeation enhancers.

Although, it may not generally be necessary to incorporate permeation enhancers in medicaments of the invention for use at sites of existing wounds, there may be circumstances in which it will be desirable to use such agents, for instance to hasten uptake of the active compound from the medicament.

Experiments undertaken by Mahmoud and co-workers using a reconstructed epidermis model to assess uptake through the skin (i.e. transdermally) indicate that application of 100 μg of oestradiol in a gel leads to 15.7 μg of the applied oestradiol being found in the skin or having passed through the skin in six hours from application. Accordingly in order to obtain uptake of a therapeutically effective amount of 400 ng of oestradiol it would be necessary to apply to 2.56 μg of oestradiol to the epidermal surface (for example over 1 cm² of the epidermal surface). The use of such a gel able to provide a therapeutically effective amount of an active agent through the epidermis constitutes a preferred embodiment of the invention, and may be particularly useful in the context of prophylactic use of the medicaments or methods of the invention.

Preferably the promotion of wound contraction using the medicaments or method of the invention may give rise to a healing time 1 day, 2 days, or 3 days faster than that occurring in a control-treated or untreated wound. Healing time may be calculated as the time elapsing between formation of a wound and complete closure of the wound (i.e. the point at which wound width becomes zero, or imperceptible). More preferably the promotion of wound contraction in accordance with the invention may give rise to a time to a healing time that is at least 4 days, 5 days or 6 days faster than that occurring in a control-treated or untreated wound. It is even more preferred that promotion of wound contraction may give rise to a healing time that is at least 7 days, 8 days or 9 days faster than that occurring in a control-treated or untreated wound, and most preferably promotion of wound contraction may give rise to a time to wound closure that is at least 10 days or greater than that occurring in a control-treated or untreated wound.

It will be appreciated that certain wounds, in particular long term chronic wounds, may not heal to full closure without clinical intervention, instead reaching a point where no increase in healing occurs with time. In such cases, the time taken to arrive at the point at which no further healing occurs may be considered to constitute the time to constructive wound closure.

The inventors have found that the medicaments and methods of the invention are able to promote contraction of wounds when used either prior to the formation of a wound, or when used after a wound has already been formed. The use of the medicaments and methods of the invention prior to formation of a wound (in which case it is believed that the action of the medicament or method is to “prime” the site where wounding will occur so that wound contraction is promoted immediately upon formation of the wound) is referred to as “prophylactic use” for the purposes of the present disclosure.

The prophylactic use of agents in accordance with the invention to promote the contraction of wounds is a preferred mode of use in accordance with the invention. It will be appreciated that such use is most suitable in the case where the time and location of prospective wound formation is known, and may be particularly suitable for promoting the contraction of wounds associated with surgical procedures. However, prophylactic use of the medicaments or methods of the invention may also be of use in situations where there is an increased likelihood of wounding occurring. The inventors have found that administration of agents in accordance with the invention immediately prior to formation of a wound (e.g. in the hour preceding wounding, or preferably in the forty minutes or thirty minutes preceding wounding, and more preferably in the ten minutes preceding wounding) is highly effective, though administration at earlier times (e.g. up to 24 or 48 hours before wounding) may also beneficially promote the contraction of wounds. The prophylactic use of methods and medicaments of the invention is a preferred embodiment of the invention, and is particularly preferred in the event that it is wished to promote contraction of surgical wounds.

Injection, and particularly intradermal injection, constitutes a preferred manner in which the medicaments of the invention may be administered (or the methods of the invention effected), as considered elsewhere in the specification. In the case of prophylactic use, it may be particularly preferred that a medicament of the invention be administered by intradermal injection to a site where wounding will take place. If the medicament is administered only a short time prior to wound, then intradermal injection of this type will typically lead to the formation of a raised bleb which will remain at the time of wounding. A wound may then be formed through the bleb. Wounds formed in this way will benefit from promotion of wound contraction in accordance with the present invention. Alternatively, blebs formed by intradermal injection of medicaments of the invention may be allowed to resolve before a wound is formed.

The medicaments and methods of the invention may also be used to promote wound contraction once the wound in question has already been formed. This use will be the use generally adopted in respect of accidental wounds and/or chronic wounds (and indeed most wounds formed other than in association with a surgical procedure).

When used to treat existing wounds medicaments in accordance with the invention may preferably be injected along the margins of wounds to be treated. Injection in this manner also constitutes a preferred route of administration in accordance with the methods of treatment of the invention. In the case of skin wounds it is preferred that the route of injection selected is intradermal injection.

In the event that the medicaments or methods of the invention are to be used to promote the contraction of an existing wound, it is preferred that such use should occur as early as possible after formation of the wound. That said, the medicaments or methods of the invention may help to promote the contraction of a wound if used at any time up until full healing has occurred (for example even if administered to a partially healed wound the medicaments of the invention may promote the contraction of the wound in a manner that will therapeutically decrease the time until closure of the wound).

In the case of wounds that are incapable of healing fully without surgical intervention (such as grafting or suturing), the medicaments or methods of the invention may be of benefit if used at any time up until the point when such intervention occurs.

Factors that may be considered in relation to the “window” in which medicaments or methods of the invention may be beneficially employed such that they are able to therapeutically influence the contraction of wounds will include: the nature of the wound in question (for example: is the wound at a site that is generally subject to “fast” or “slow” healing?); the severity of the wound (what is the extent of the damage that has occurred?); and the size of the damaged area. Thus in the case of a wound of large area, or in a site that is naturally associated with slower than average healing, the methods or medicaments of the invention may be still be effective to therapeutically promote the contraction of the wound even if administered relatively late in the healing response. Thus, although the medicaments or methods of the invention may preferably be administered within the first one to 24 hours after formation of an acute wound, beneficial promotion of wound contraction may also be brought about if administered up to ten, or more, days after the wound is formed.

It will be appreciated that in the case of chronic wounds, the period in which the medicaments or methods of the invention may be beneficially employed will be considerably longer. Chronic wounds may persist for many years, and the healing of wounds that may be many years old may be beneficially promoted through promotion of wound contraction using the medicaments or methods of the invention.

Therapeutic promotion of the contraction of wounds may be achieved using only a single administration of the medicaments or methods of the invention. Due to the simplicity of this therapeutic regime it constitutes a preferred use of the medicaments and methods of the invention.

However, there may be cases in which it is preferred that the medicaments or methods of the invention be used in repeated incidences of therapy. Thus treatment to promote contraction of a wound may involve administration of medicaments of the invention on more than once occasion. Use in this manner may be preferred in the case of large wounds, or of wounds that are resistant to treatment, or subject to retarded healing (such as chronic wounds). Generally medicaments of the invention may be administered to a wound as required until therapeutically effective wound contraction has been achieved (for example, until the wound has closed, or no further contraction of the wound may be promoted). By way of example medicaments of the invention may be administered daily (or on multiple occasions within a given day), or may be administered after a delay of multiple days.

Generally when the medicaments or methods of the invention are to be used in multiple therapeutic incidences, administration should be repeated until contraction of a wound has been promoted to a clinician's satisfaction.

It may be preferred that the medicaments or methods of the invention are utilised both before and after wounding.

It may be preferred that the medicaments of the invention are administered to a site where they are to have their effect around the time of wounding, or immediately prior to the forming of a wound (for example in the period up to six hours before wounding, and particularly in the period of 10, 20, 40 or 60 minutes prior to wound formation) or the medicaments may be administered at an earlier time before wounding (for example up to 48 hours before a wound is formed). The skilled person will appreciate that the most preferred times of administration prior to formation of a wound will be determined with reference to a number of factors, including the formulation and route of administration of the selected medicament, the dosage of the medicament to be administered, the size and nature of the wound to be formed, and the biological status of the patient (which may be determined with reference to factors such as the patient's age, health, and predisposition to healing complications or adverse scarring) and the half-life of the compound that promotes oestrogenic activity in the body.

It may be particularly preferred that the methods or medicaments of the invention may be administered both before and after formation of a wound. The inventors believe that administration of the medicaments of the invention immediately prior to the formation of a wound, followed by administration of a compound that promotes oestrogenic activity for one or more days following wounding, is particularly effective in promoting wound contraction.

For the purposes of the present specification, an “agent” or “agent of the invention” will be a therapeutically effective compound able to promote oestrogenic activity. It will be appreciated that all such suitable agents may be incorporated in medicaments in accordance with the invention, and all may be used in the methods or uses of the invention. The medicaments of the invention represent preferred compositions by which a therapeutically effective amount of a compound that promotes oestrogenic activity may be administered in order to put the methods of the invention into practice. 17β-oestradiol is a preferred example of an agent, or agent of the invention, as considered elsewhere in the specification.

It will be appreciated that the amount of a medicament of the invention that should be provided to a wound, in order that a therapeutically effective amount of compound that promotes oestrogenic activity may be administered, depends on a number of factors. A number of these are discussed elsewhere in the specification, and include the biological activity and bioavailability of the agent present in the medicament, which in turn depends, among other factors, on the nature of the agent and the mode of administration of the medicament. Other factors in determining a suitable therapeutic amount of a medicament may include:
A) The half-life of the agent in the subject being treated.
B) The specific wound to be treated (e.g. promoting contraction of an acute wound or a chronic wound).
C) The age of the subject.
D) The size of the site to be treated.

The frequency of administration will also be influenced by the above-mentioned factors and particularly the half-life of the chosen agent within the subject being treated.

Frequency of administration will depend upon the biological half-life of the agent used. Typically a cream or ointment containing an agent of the invention should be administered to a target tissue such that the concentration of the agent at a wound is maintained at a level suitable to promote wound contraction. This may be achieved by a single administration of a composition incorporating a compound that promotes oestrogenic activity, or may require administration of such a composition daily or even several times daily.

Medicaments of the invention, may be administered by any suitable route capable of achieving the desired effect of promoting wound contraction, but it is preferred that the medicaments be administered locally at a wound site or site where a wound is to be formed.

Administration (and particularly topical administration) of the medicaments of the invention may be effected as part of the initial and/or follow up care for the wounded area.

As suggested elsewhere in the specification, the agents of the invention may be provided on a dressing or patch, which may be used to cover a wound the contraction of which is to be promoted. It will be appreciated that such a dressing or patch used to administer an agent of the invention may preferably be provided in a sterile form.

The agents of the invention may be released from a device or implant, or may be used to coat such a device e.g. a stent or controlled release device.

It will be appreciated that the vehicle of a composition comprising agents of the invention should be one that is well tolerated by the patient and allows release of the agent to the wound to which the composition is provided. Such a vehicle is preferably biodegradable, biocompatible, bioresolveable, bioresorbable and/or non-inflammatory. If the composition is to be applied to an existing wound then the pharmaceutically acceptable vehicle will be one that is relatively “mild”.

Delayed release devices may be particularly useful for patients requiring protracted treatment with the medicaments or methods of the invention, such as those requiring therapeutic contraction of chronic wounds. Delayed release devices may be particularly advantageous when used for the administration of an agent that would otherwise normally require frequent administration (e.g. at least daily administration by other routes).

Daily doses of an agent of the invention may be given as a single administration (e.g. a daily application of a topical formulation or a daily injection). Alternatively, the agent of the invention may require administration twice or more times during a day. Each such administration may provide a therapeutically effective amount of the agent, or a known fraction of such a therapeutically effective amount. In a further alternative, a slow release device may be used to provide optimal doses of an agent of the invention to a patient without the need to administer repeated doses.

A dose of a composition comprising agents of the invention may preferably be sufficient to provide a therapeutically effective amount of compound that promotes oestrogenic activity in a single administration. However, it will be appreciated that each dose need not in itself provide a therapeutically effective amount of a compound that promotes oestrogenic activity, but that a therapeutically effective amount may instead be built up through repeated administration of suitable doses.

Various suitable forms are known for compositions comprising agents of the invention. In one embodiment a pharmaceutical vehicle for administration of an agent of the invention may be a liquid and a suitable pharmaceutical composition would be in the form of a solution. Such a solution may be administered by injection, or by other routes, such as by a spray. An agent of the invention may be formulated as part of a cream, gel or ointment. In a further embodiment the agent of the invention may be formulated as a part of a pharmaceutically acceptable patch providing delivery of a therapeutically effective amount of a compound that promotes oestrogenic activity to a wound or a site where a wound is to be formed.

A solid vehicle can include one or more substances that may also act as lubricants, solubilizers, suspending agents, fillers, glidants, compression aids, or binders; it can also comprise an encapsulating material. In powders, the vehicle is a finely divided solid that is in admixture with the finely divided active agent. Powders may preferably contain up to 99% of the active agent. Suitable solid vehicles include, for example, calcium phosphate, magnesium stearate, talc, sugars, lactose, dextrin, starch, gelatin, cellulose, polyvinylpyrrolidine, low melting waxes and ion exchange resins.

Liquid vehicles may be used in preparing solutions, suspensions, emulsions, syrups, elixirs and pressurized compositions (sprays). The agent of the invention can be dissolved or suspended in a pharmaceutically acceptable liquid vehicle such as water, an organic solvent, a mixture of both or pharmaceutically acceptable oils or fats. It may generally be preferred that the medicaments or methods of the invention make use of oestrogenic compounds diluted in an aqueous solution (i.e. a solution in which water is the major diluent). It may be preferred that medicaments or methods of the invention make use of oestrogenic compounds dissolved in diluents other than oils. In the case where it is desired to utilise an oil as a diluent in accordance with a medicament or method of the invention it may generally be preferred to use synthetic oils or mineral oils. Suitable diluents, whether aqueous or otherwise, may preferably be substantially free from allergens or toxins, such as those that may be found in naturally occurring products (such as nut or vegetable oils) that may otherwise be used as diluents in medicaments or methods of the invention. It will be appreciated that the considerations above are of particular concern in the case of medicaments or methods of the invention in which oestrogenic compounds are administered by means of injection. Furthermore, the inventors have found that the use of oil-based diluents in the preparation of compositions for administration by localised injection (such as intradermal injection) to wounds, or sites where wounds are to be formed, may generally be disadvantageous, and can give rise to deleterious effects that may retard the wound healing process and lead to the production of abnormally wide wounds. Thus it may be preferred not to use oils in the preparation of injectable medicaments in accordance with the invention.

The liquid vehicle can contain other suitable pharmaceutical additives such as solubilizers, emulsifiers, buffers, preservatives, sweeteners, flavouring agents, suspending agents, thickening agents, colours, viscosity regulators, stabilizers or osmo-regulators. Suitable examples of liquid vehicles for parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). A solution comprising 95% phosphate buffered saline (PBS) and 5% ethanol is a particularly preferred vehicle for use in compositions comprising 17β-oestradiol as the agent of the invention. For parenteral administration, the vehicle can be an oily ester such as ethyl oleate and isopropyl myristate. Sterile liquid vehicles are useful in sterile liquid form compositions for parenteral administration. The liquid vehicle for pressurized compositions can be halogenated hydrocarbon or other pharmaceutically acceptable propellant.

Liquid pharmaceutical compositions which are sterile solutions or suspensions can be utilized by, for example, intramuscular, intraperitoneal, intradermal, intraadventitial (blood vessels) or subcutaneous injection. Sterile solutions can also be administered intravenously (for instance in the case where it is wished to promote contraction of wounds in blood vessels). The agent of the invention may be prepared as a sterile solid composition that may be dissolved or suspended at the time of administration using sterile water, saline, or other appropriate sterile injectable medium (such as PBS). Vehicles are intended to include necessary and inert binders, suspending agents, lubricants and preservatives.

Agents of the invention may be used to promote contraction of “internal” wounds (i.e. wounds occurring within the body, rather than on an external surface of the body). Examples of internal wounds include penetrative wounds that pass through the skin into underlying tissues, and wounds associated with surgical procedures conducted within the body.

It will be appreciated that the use of medicaments or methods of the invention to promote the contraction of internal wounds will necessitate the use of suitable routes of administration, thereby requiring the formulation of the agents of the invention in a manner that allows their delivery to the wound in question. For example, medicaments in accordance with the invention for promoting the contraction of wounds in the lungs or other respiratory tissues may be formulated for inhalation. In another preferred embodiment, medicaments in accordance with the invention for promoting the contraction of wounds in the body cavities (such as the abdomen or pelvis) may be formulated as a lavage, gel or instillate.

Known procedures, such as those conventionally employed by the pharmaceutical industry (e.g. in vivo experimentation, clinical trials etc), may be used to establish specific formulations of compositions comprising agents of the invention and precise therapeutic regimes for administration of such compositions (such as daily doses of the active agent and the frequency of administration).

The inventors believe that the amount of a compound that promotes oestrogenic activity (such as 17β-oestradiol) that may be administered to a wound (or site where a wound is to be formed) in a single incidence of treatment may be formulated to deliver approximately 1.47 nmoles of the compound per centimetre of wound, or per cm² of a site where a wound is to be formed.

In the event that multiple administration of a compound that promotes oestrogenic activity is to be utilised, the preferred amount of compound that promotes oestrogenic activity to be administered to a wound (or site where a wound is to be formed) over a period of approximately 24 hours may be in the region of 1.47 nmoles per centimetre of wound (or per cm² of a site where a wound is to be formed).

The skilled person will appreciate that the suggestions above are provided for guidance. In particular it will be appreciated that the amount of a compound that promotes oestrogenic activity to be administered via topical administration may be altered depending on permeability of the tissue or organ to which the topical composition is administered. Thus in the case of relatively impermeable tissues or organs it may be preferred to increase the amount of the agent administered. Such an increased amount of a compound that promotes oestrogenic activity may still represent a therapeutically effective amount, if the amount of the agent taken up into the tissue or organ where wound contraction is to be promoted is therapeutically effective (i.e. if a therapeutically effective amount permeates the tissue or organ where wound contraction is to be promoted, irrespective of the fact that a larger amount of the agent may remain on the surface of, and unable to penetrate, the tissue or organ being treated).

In a particularly preferred embodiment, the compound that promotes oestrogenic activity (for instance 17β-oestradiol) may be administered as a 14.7 μM solution, with 100 μL of such a solution administered per centimetre of wound over a 24 hour period.

The skilled person will appreciate that effective therapeutic amounts of a compound that promotes oestrogenic activity may be determined with reference to the concentration of the agent that is attained in the organ or tissue to which they are administered. The information regarding therapeutically effective dosages set out herein will provide sufficient guidance to allow the skilled person to calculate the local concentrations of a compound that promotes oestrogenic activity (such as 17β-oestradiol) established by intradermal injection, and, based on these values, to determine suitable amounts of such agents that may be administered by other routes in order to achieve equivalent local concentrations. It will be appreciated that the tissue concentration of a compound that promotes oestrogenic activity to be established should be one at which the selected compound achieves the therapeutically effective level of oestrogenic activity required.

The inventors have found that 17β-oestradiol may be administered by way of an injectable solution containing approximately 400 ng/100 μL in order to promote wound contraction when administered as an intradermal injection providing 100 μL of solution per cm of wound.

It will be appreciated that the guidance as to doses and amounts of agents that promote oestrogenic activity to be used provided above is applicable both to medicaments of the invention, and also to the methods of the invention.

In the case where the paragraphs above consider the administration of a specified amount of a medicament per cm of a wound it will be appreciated that this volume may be administered to either one or both of the margins of a wound to be treated (i.e. in the case of a reference to 100 μl of a medicament, this may be administered as 100 μalong one of two wound margins to be joined together, or as 50 μl to each of the wound margins to be joined together).

Medicaments of the invention may be used to promote wound contraction as a monotherapy (e.g. through use of medicaments of the invention alone). Alternatively the methods or medicaments of the invention may be used in combination with other compounds or treatments for the promotion of wound contraction, or other means by which healing of a wound may be promoted. Suitable compounds that may be used as parts of such combination therapies will be well known to those skilled in the art.

Although it is preferred that the medicaments or methods of the invention be used in human patients, it will be appreciated that many of the advantages that may be gained as a result of promotion of the contraction of human wounds are also are also applicable to wounds in other animals, particularly veterinary or domestic animals (e.g. horses, cattle, dogs, cats etc). Accordingly it will be recognised that the medicaments and methods of the invention may also be used to promote the contraction of wounds of non-human animals.

The invention will now be further described with reference to the accompanying Experimental Results and Figures, in which:

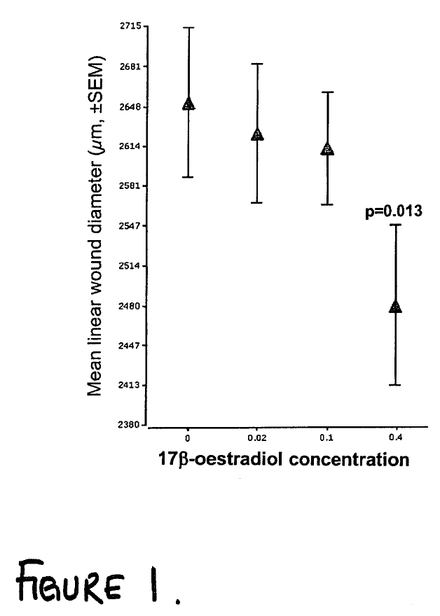

FIG. 1 illustrates the results of a study comparing wound width three days after punch biopsies in human volunteers, and shows that medicaments of the invention are able to statistically significantly promote contraction of treated wounds, as compared to untreated or control treated wounds. 17β-oestradiol concentration is shown as the amount present in a 100 μl intradermal injection of a solution comprising 17β-oestradiol in a diluent of PBS and 5% v/v ethanol.

### Experimental Results

Medicaments of the invention promote contraction of full thickness cutaneous excisional wounds (punch biopsies) in humans

### Materials and Methods

A single site, double-blind study was undertaken to illustrate the efficacy of medicaments of the invention in promoting contraction of punch biopsy wounds in healthy male and post-menopausal female subjects. The control treatments used in this study were Placebo (vehicle) and Standard Care (moist wound healing dressings).

44 healthy subjects were recruited to the study, of whom, 40 were healthy males (aged 18-75years) and 4 were post-menopausal females (aged 53-69 years). The average age of subjects recruited to this study was 42 years. The post-menopausal status of female subjects was confirmed by quantification of serum levels of estradiol and follicle stimulating hormone (FSH). Serum levels of estradiol were required to be <90 pmol/L, and follicle stimulating hormone levels >31 IU/L.

Before wounding, each subject received either a medicament of the invention (providing a 100 μl intradermal injection of a solution comprising 400 ng of 17β-oestradiol in a diluent comprising PBS and 5% v/v ethanol), or a control medicament (two used, providing a 100 μl intradermal injection of a solution comprising either 20 ng or 100 ng respectively of 17β-oestradiol in a diluent comprising PBS and 5% v/v ethanol). The experimental medicaments (either of the invention or control) were administered intradermally as a single dose at a volume of 100 μl per wound site, 10-30 minutes before wounding to sites on the upper, inner aspect of the arm. This administration was sufficient to prophylactically “prime” an area of 1 cm² of unwounded skin prior to wounding. Wounds were made with a 3 mm punch biopsy.

Biopsy sites were excised, using a 5 mm punch biopsy, three days after wounding. Excised wounds were preserved for histological assessment of wound width. All histological assessments were made using preserved 5 micron-thickness wound sections taken from the widest part of each excised biopsy site. Sections were stained with Haematoxylin and Eosin to aid visualisation of structural features, and measurements made using image analysis software. The wound diameter is the linear total wound diameter of the wound section measured in micrometres. The average wound diameter value was calculated from the 44 wounds of each treatment group.

### Results

Wound diameters of the treated wound, control treated wounds and untreated wounds were determined using the methods set out above.

Untreated control wounds receiving standard care had an average wound diameter of 2652 μm; control treated wounds receiving 20 ng of 17β-oestradiol had an average wound width of 2626 μm, and control treated wounds receiving 100 ng of 17β-oestradiol had an average wound width of 2612 μm.

In contrast, wounds treated with the medicament of the invention (providing 400 ng of 17β-oestradiol) had an average wound width of 2480 μm, significantly decreased compared to the widths of control treated or untreated wounds.

These results clearly illustrate that the medicaments and methods of the invention are able to promote wound contraction, and thereby significantly decrease wound width, as compared to control treatments. This is particularly effective using medicaments that provide oestrogenic activity corresponding to that produced by approximately 400 ng of 17β-oestradiol per cm² of unwounded skin (or centimetre of wounding) to which they are administered.

1. The use of a compound that promotes oestrogenic activity in the manufacture of a medicament for the promotion of wound contraction.

2. The use according to claim 1, wherein the medicament provides an amount of the compound that promotes oestrogenic activity equivalent to that promoted by approximately 400 ng of 17β-oestradiol, per centimetre of wound, or cm² of unwounded tissue, to which the medicament is administered.

3. The use according to claim 1, wherein the compound that promotes oestrogenic activity is selected from the group consisting of: oestrogens; oestrogen receptor agonists such as ethinylyoestradiol, dienoestrol, mestranol, oestradiol, oestriol, conjugated oestrogens, piperazine oestrone sulphate, stilboestrol, fosfesterol tetrasodium, polyestradiol phosphate and tibolone;. inhibitors of oestrogen or oestrogen receptor agonist breakdown; phytoestrogens; modulators of luteinising hormone; and chorionic gonadotrophin; and 17βoestradiol.

4. The use according to claim 1, wherein the compound that promotes oestrogenic activity is 17β-oestradiol.

5. The use according to claim 4, wherein the medicament is formulated to provide approximately 400 ng of 17β-oestradiol, per centimetre of wound.

6. The use according to claim 1, wherein the wound is selected from the group consisting of: superficial injuries, abrasions, cuts, pressure ulcers stages i) to iv); venous stasis ulcers, ulcers caused by mixed etiologies; lower extremity ulcers; diabetic ulcers; radiation ulcers; arterial ulcers; partial thickness excisions; full thickness excisions; wounds in the immunocompromised, elderly or paraplegics; pre-tibial lacerations; wounds that have been debrided; surgical incisions; and surgical excisions.

7. The use according to claim 1, wherein the wound is a skin wound.

8. The use according to claim 1, wherein the medicament is a topical medicament.

9. The use according to claim 1, wherein the medicament is a liquid medicament.

10. The use according to claim 9, wherein the liquid medicament is selected from the group consisting of: gels: thermosetting gels; creams; ointments; sprays; injectable solutions; irrigation solutions; other solutions of oestrogenic compounds; and liquid compositions comprising microparticles, nanoparticles or liposomes.

11. The use according to claim 10, wherein the medicament is for intradermal injection.

12. The use according to claim 1, wherein the medicament is a solid medicament.

13. The use according to claim 12, wherein the solid medicament is selected from the group consisting of: powders; sprays, crystals; microneedles; solid compositions comprising microparticles, nanoparticles or liposomes; and wound dressings, including foam or sponge dressings, or film dressings.

14. The use according to claim 13, wherein the medicament comprises a wound dressing.

15. The use according to claim 1, wherein the medicament is for prophylactic use.

16. The use according to claim 1, wherein the pattern of administration of the medicament comprises administration of the medicament between 10 minutes and 40 minutes prior to wounding.

17. The use according to claim 1, wherein the medicament is for administration to an existing wound.
